(19) United States
(12) Patent Application Publication
(10) Pub. No.: US 2018/0125449 A1
(43) Pub. Date: May 10, 2018

(54) Ultrasound Imaging System with Mechanical Support Structure

(71) Applicant: Rivanna Medical LLC, Charlottesville, VA (US)

(72) Inventors: Frank William Mauldin, JR., Charlottesville, VA (US); Kevin Owen, Crozet, VA (US); Adam Dixon, Charlottesville, VA (US)

(21) Appl. No.: 15/867,051

(22) Filed: Jan. 10, 2018

Related U.S. Application Data
(63) Continuation-in-part of application No. 14/770,896, filed on Aug. 27, 2015.
(60) Provisional application No. 61/770,448, filed on Feb. 28, 2013.

(51) Int. Cl. A61B 8/08; A61B 8/00

(52) U.S. Cl. CPC A61B 8/0841; A61B 8/462; A61B 8/4427; A61B 8/469; A61B 2017/3413; A61B 8/488; A61B 8/483; A61B 8/5207; A61B 8/4455

(57) ABSTRACT

In at least some aspects, a handheld ultrasound imaging device comprises: a housing; an ultrasound imaging unit disposed at least in part within the housing, the ultrasound imaging unit including an ultrasound transducer; a display rotatably coupled to the housing; and a releasably engageable catch; wherein when the catch is releasably engaged, the catch restricts movement of the housing relative to the display. In at least some aspects, a method comprises: positioning a handheld ultrasound imaging device having a housing and a display on a surface with a screen of the display facing toward the surface; providing relative movement between the housing and display to position an ultrasonic transducer of the handheld ultrasound imaging device in an at least partially upright position; and applying a sterile cover on at least a portion of the ultrasonic transducer without touching the handheld ultrasound imaging device.

### RELATED APPLICATIONS

This application is a continuation in part of U.S. patent application Ser. No. 14/770,896, filed on Aug. 27, 2015, entitled “Localization of Imaging Target Regions and Associated Systems and Devices,” which is a U.S. National Stage application under 35 U.S.C. 371 of International Application No. PCT/US2013/077917, filed on Dec. 27, 2013, which claims the benefit of and priority to Provisional Application No. 61/770,448, filed on Feb. 28, 2013. Each of the foregoing are incorporated by reference herein in their entirety.

### GOVERNMENT SPONSORSHIP

This invention was made with government support under award number R43EB015232 awarded by the National Institute of Biomedical Imaging And Bioengineering of the National Institutes of Health and award number 1214788 awarded by the National Science Foundation. The government has certain rights in this invention.

### TECHNICAL FIELD

Imaging devices are generally described.

### BACKGROUND

Medical ultrasound is a popular medical imaging modality primarily used for diagnostic imaging of soft tissue but also for interventional procedures such as guidance of a needle or catheter placement. Examples include diagnostic imaging of organs, such cardiac or liver structures. Common interventional procedures that rely on ultrasound guidance are central line placement and guidance of nerve blocks, both of which are high volume procedures in certain hospital settings such as the intensive care unit (ICU). Current ultrasound systems are mostly cart-based and optimized for superior contrast and resolution in soft tissue. However, these systems are generally expensive, and in a hospital setting with multiple physicians per ultrasound system, they can be difficult to access.

### SUMMARY

Handheld ultrasound imaging devices are generally described. Certain embodiments have improved portability relative to prior imaging systems. In addition, attachable accessories that facilitate interventional procedures (including interventional ultrasound procedures) involving directing a probe (e.g., needle or catheter) to a probe target (e.g., blood vessel or nerve bundle) are also described. Certain embodiments can be used in handheld imaging devices, which address many of the limitations of prior art systems by, for example, providing standard imaging capabilities in a low cost portable device. The subject matter of the present invention involves, in some cases, interrelated products, alternative solutions to a particular problem, and/or a plurality of different uses of one or more systems and/or articles.

In one aspect, a handheld ultrasound-imaging device is provided. In certain embodiments, the handheld ultrasound imaging device comprises a housing comprising a first end comprising an ultrasound imaging unit, a second end comprising a display, and a hand grip region between the ultrasound imaging unit and the display.

In certain embodiments, the handheld ultrasound imaging device comprises a housing comprising an ultrasound imaging unit, and a marking unit detachably coupled to the housing and comprising a probe indicator configured to indicate proper placement of a probe at or near a target that is to be imaged, and positioned such that, during use, aline extending between the target and the ultrasound imaging unit intersects the marking unit.

The handheld ultrasound-imaging device comprises, according to some embodiments, a housing comprising an ultrasound imaging unit, a handgrip region, and a rotatable display, wherein the rotatable display is configured to be rotated, relative to at least another portion of the housing, about at least one rotational axis by at least about 30 degrees.

Any of the above handheld ultrasound imaging devices may comprise, according to some embodiments, an actuator on the handgrip region configured such that when the actuator is activated, data from the ultrasound-imaging unit is recorded and/or manipulated. The actuator may be, according to certain embodiments, configured to perform one or more of a number of other functions including, but not limited to, video save, device power on/off, image settings adjustment (e.g., imaging mode, gain, frequency, contrast, depth), and/or menu navigation.

Any of the above handheld ultrasound imaging device may comprise, according to certain embodiments, a marking unit comprising a vacuum-generating unit configured to adhere the marking unit to a surface associated with the target.

In one aspect, imaging devices are provided. In one set of embodiments, the imaging device comprises a housing comprising an imaging unit and a handle; and a rotatable display connected to the housing, wherein the rotatable display is configured to be rotated about at least one rotational axis by at least about 30 degrees. The imaging device can be configured, in some embodiments, to have one or more of the following properties. In some embodiments, the imaging device is configured to produce an image along a scanning plane. In certain embodiments, the housing is elongated and comprises a longitudinal axis. In some embodiments, the smallest angle between the scanning plane of the imaging unit and the longitudinal axis of the housing is less than about 45°.

In certain embodiments, the scanning plane of the imaging unit is substantially parallel to the longitudinal axis of the housing. According to certain embodiments, the rotatable display is configured to be rotated such that the display can be oriented in a first position substantially parallel to a scanning plane of the imaging unit and in a second position substantially perpendicular to the scanning plane of the imaging unit. In some embodiments, the handle is located directly above the imaging unit during operation of the imaging device. The imaging device can be a handheld imaging device, according to certain embodiments. In some embodiments, the imaging device occupies a volume of equal to or less than about 500 cm³. The imaging unit can comprise, according to certain embodiments, an ultrasound transducer.

In certain embodiments, the imaging device comprises a handheld self-contained ultrasound imaging device comprising a display directly attached to the top of a housing, an ultrasound transducer positioned at the bottom of the housing, and a handle incorporated with the housing and positioned between the display and the ultrasound transducer, wherein the display is rotatable about at least one rotational axis by at least about 30°.

In another aspect, a cover for an imaging device is provided. The cover comprises, in certain embodiments, a cover body configured to be attached to an imagingdevice and a marking unit attached to the cover body and configured to indicate proper placement of a probe along a target that is to be imaged. The cover can have any one or more of the following properties, according to various embodiments. In some embodiments, the cover is configured such that when it is attached to the imaging device, an image can be produced without the use of transmission medium. For example, in certain embodiments, the cover is configured such that when it is attached to an ultrasound-imaging device, an image can be produced without the use of ultrasound gel or other ultrasound transmission medium. In some embodiments, the marking unit is configured to identify a target surface location corresponding to a center of an imagingscan plane. In some embodiments, the marking unit comprises an identifying mark indicating the target surface location corresponding to a center of an imaging scan plane. In certain embodiments, the identifying mark comprises a hole. In some embodiments, the marking unit comprises an adhesive material configured to adhere the marking unit to a surface of a target. In some embodiments, the marking unit comprises a moveable tab configured to indicate a site corresponding to a center of a scan plane. In certain embodiments, the moveable tab is configured to make a visible indentation at a site corresponding to the center of the scan plane. The probe can be, in certain embodiments, a needle and/or a catheter. For example, according to certain embodiments, the marking unit attached to the cover body is configured to indicate proper placement of a needle or a catheter. The cover can be, in certain embodiments, configured to be attached to an ultrasound imaging device.

Many ultrasound procedures require that ultrasound scanning be performed in a sterile manner. This is especially true for ultrasound guidance of needle or catheter placements or intraoperative ultrasound. Current ultrasound systems are made sterile by placing a sterile sleeve over the ultrasound transducer. This currently requires two people. A first person, who is wearing sterile gloves, holds a sterile sleeve, while a second person places a non-sterile ultrasound transducer into the sterile sleeve. In the course of such process, the non-sterile ultrasound transducer and the person holding it must not touch the person wearing sterile gloves.

However, it has been determined that a handheld ultrasound-imaging device may be provided with a configuration that allows one person to apply a sterile sleeve or other type of sterile cover over the ultrasound transducer, at least in part, without help from another person.

In at least some embodiments, such a configuration and method is beneficial and less time and resource consuming than current systems and methods.

In that regard, in at least some embodiments, a handheld ultrasound-imaging device may have a housing (for an ultrasound transducer), a rotatable display and a configuration that allows the rotatable-display (a housing thereof or otherwise) to be used as a stand, and further allows the ultrasound transducer to be positioned at least partly upright, with a membrane of the ultrasound transducer facing up. The device further includes a brace to resist (to at least some degree) movement of the housing relative to the rotatable-display.

With such configuration, the handheld ultrasound-imaging device may be positioned on a surface (non-sterile or otherwise), a gel or other ultrasound transmission medium may be applied to the ultrasound transducer without falling off, and a person wearing sterile gloves may apply a sterile cover on the ultrasound transducer, at least in part, while touching only the sterile cover.

In at least some aspects, a handheld ultrasound imaging device comprises: a housing; an ultrasound imaging unit disposed at least in part within the housing, the ultrasound imaging unit including an ultrasound transducer; a display rotatably coupled to the housing; and a releasably engageable catch; wherein when the catch is releasably engaged, the catch restricts movement of the housing relative to the display.

In at least some embodiments, when the catch is releasably engaged, the catch restricts the housing to at least partly upright positions relative to the display.

In at least some embodiments, when the catch is releasably engaged, the catch resists all movement of the housing relative to the display.

In at least some embodiments, the catch comprises a detent.

In at least some embodiments, the catch is release by application of catch release force to at least one of the housing and the stand.

In at least some embodiments, the handheld ultrasound imaging device further comprises a button coupled to the catch and depressible to release the catch.

In at least some aspects, a handheld ultrasound imaging device comprises a housing; an ultrasound imaging unit disposed at least in part within the housing, the ultrasound imaging unit including an ultrasound transducer; a display; a pivot, the housing and display rotatably coupled through the pivot; and a brace external to said pivot to provide a friction force that resists rotation between the housing and the display.

In at least some embodiments, the brace has a brace surface external to said pivot and in contact with a surface that moves relative to the brace surface with rotation between the housing and the display to provide the friction force that resists rotation between the housing and the display.

In at least some embodiments, the brace surface and the surface that moves relative to the brace surface define an interface having a press fit.

In at least some embodiments, the brace surface comprises a concave surface and wherein the surface that moves relative to the brace surface comprises a convex surface.

In at least some aspect, a method comprises: positioning a handheld ultrasound imaging device having a housing and a display on a surface with a screen of the display facing toward the surface; providing relative movement between the housing and display to position an ultrasonic transducer of the handheld ultrasound imaging device in an at least partially upright position; and applying a sterile cover on at least a portion of the ultrasonic transducer without touching the handheld ultrasound imaging device.

In at least some embodiments, the method further comprises applying a transmission medium on a surface of the ultrasonic transducer.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic illustration of an exemplary imaging device, according to certain embodiments;

FIGS. 2A-2C are, according to one set of embodiments, a series of schematic illustrations outlining the rotation of a display of an exemplary imaging device;

FIG. 3A is a schematic illustration of an imaging device comprising an exemplary detachable marking unit, according to some embodiments;

FIG. 3B is a schematic cross-sectional illustration of a marking unit, according to certain embodiments;

FIGS. 4A-4D are a series of schematic illustrations showing the use of an exemplary detachable marking unit, according to certain embodiments;

FIGS. 5A-5E are, according to certain embodiments, a series of schematic illustrations showing the use of an exemplary detachable marking unit;

FIG. 6 is a series of schematic illustrations showing the connectivity between an exemplary detachable marking device and an imaging device, according to one set of embodiments;

FIG. 7 is a schematic illustration of an exemplary detachable marking unit comprising a needle sleeve, according to certain embodiments;

FIG. 8 is an exemplary imaging unit display, according to one set of embodiments;

FIGS. 9A-9D are schematic illustrations of a handheld ultrasound-imaging device, according to at least some embodiments;

FIG. 10 is a schematic illustration of a handheld ultrasound-imaging device, according to at least some embodiments;

FIG. 11A is a schematic illustration of a handheld ultrasound-imaging device, according to at least some embodiments;

FIG. 11B is a schematic illustration of a handheld ultrasound-imaging device, according to at least some embodiments;

FIGS. 12A-12D are a series of schematic illustrations showing a method for application of a transmission medium and sterile cover to a handheld ultrasound-imaging device, according to at least some embodiments; and

FIG. 13 is a flowchart of a method, according to at least some embodiments.

### DETAILED DESCRIPTION

Imagining devices for identification of target regions are generally described. Certain embodiments relate to handheld ultrasound imaging devices. Various embodiments are related to configurations that may provide, in some cases but not necessarily all cases, one or more advantages during operation of the imaging device.

Certain embodiments are related to handheld ultrasound imaging devices in which the imaging unit, the display, and the handgrip region are positioned relative to each other such that operation of the imaging device is made easier, under certain circumstances. In one set of embodiments, the handheld ultrasound imaging device comprises a housing, which can include an ultrasound imaging unit at one end, a display at another end, and a handgrip region between the ultrasound imaging unit and the display. As described in more detail below, arranging the imaging unit, the hand grip region, and the display in this manner can allow one to direct the movement of the handheld device relatively easily (e.g., by resting the side of the hand on a surface of the object that is being imaged) while maintaining the ability to see the display screen.

Certain embodiments are related to the use of rotatable displays in handheld ultrasound imaging devices. Generally, ultrasound-imaging devices in the past have included fixed displays or displays that are spatially separated from the housing in which the imaging unit is contained. Imaging devices with fixed displays can be difficult to operate, as they do not allow one to adjust the angle of the display relative to the eye of the user without rotating or otherwise moving the housing to which the display is connected. While imaging devices with displays that are spatially separated from the device housing allow one to adjust the position of the display relative to the imaging unit, they are generally difficult to transport and/or store when they are not in use. By integrating the display with the housing while maintaining the ability to rotate the display relative to the housing one can realize the benefit of portability and ease of storage while also allowing for relatively easy repositioning of the display relative to the users eye.

Some embodiments are directed to inventive configurations of marking units—configured to direct the placement of a probe such as a needle or catheter—in the handheld ultrasound imaging devices. For example, according to certain embodiments, a handheld ultrasound imaging device can comprise a marking unit detachably coupled to the housing of the device and positioned such that the target that is being imaged, the ultrasound-imaging unit, and the marking unit are aligned during use. Maintaining alignment of these components can allow one, according to certain embodiments, to properly align the marking unit relative to the target relatively easily during use. As described in more detail below, this alignment, according to some embodiments, may allow one to position the marking unit while the imaging unit is still in place, which can, according to certain embodiments, improve the accuracy of the placement of a probe such as a needle, a catheter, or other device.

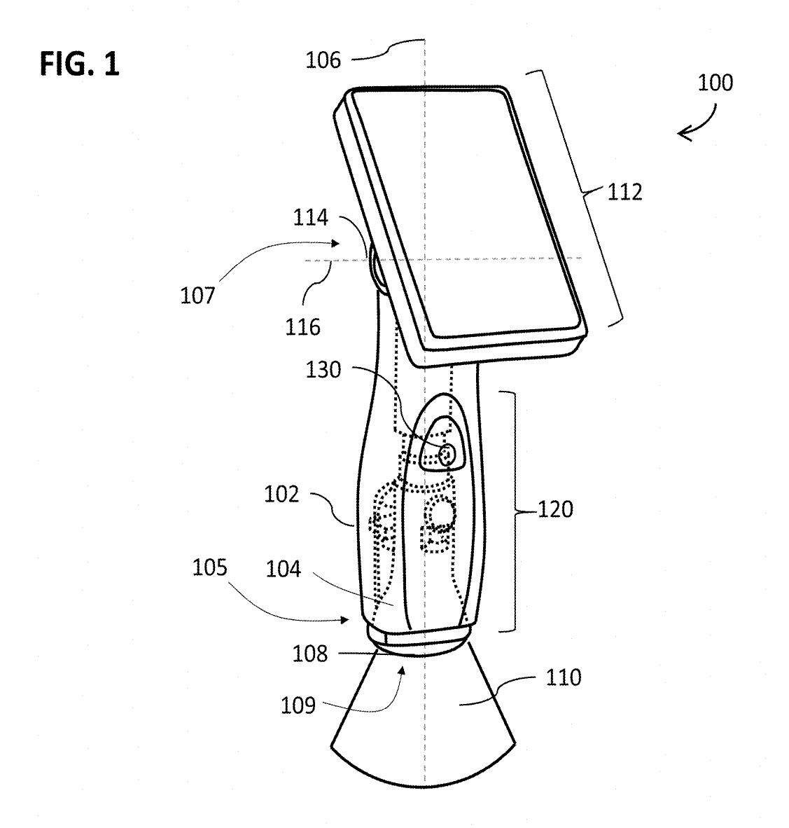

FIG. 1 is an exemplary schematic illustration of a handheld ultrasound-imaging device 100, according to certain embodiments. In certain embodiments, the handheld ultrasound imaging device comprises a housing. For example, in FIG. 1, imaging device 100 comprises housing 102. According to certain embodiments, the housing comprises a first end associated with an ultrasound-imaging unit. Referring to FIG. 1, for example, imaging unit 104 is associated with end 105 of housing 102. As shown in FIG. 1, ultrasound-imaging unit 104 is enclosed within housing 102. Other arrangements are also possible.

In some embodiments, imaging unit 104 can comprise, for example, an ultrasound transducer 108. The ultrasound-imaging unit 104 can be configured, in certain embodiments, to produce an image along at least one scanning plane 110. The imaging unit can be configured to produce an image using standard ultrasound image processing techniques known to those of ordinary skill in the art of ultrasound imaging, and described in more detail below.

The imaging device 100 comprises, in certain embodiments, display 112 (e.g., an LCD display, an OLED display, or any other suitable type of display). The display can be located at a second end of the housing. For example, in FIG. 1, display 112 is located at end 107 of housing 102. According to certain embodiments, ultrasound transducer 108 can be configured to produce data along at least one scanning plane 110 that can be subsequently output on display 112.

In certain embodiments, an ultrasound transducer surface (e.g., configured to be applied on or near a target site) can be located at one end of the housing and the display can be attached to the housing at the opposite end. For example, in FIG. 1, ultrasound transducer surface 109 is located at end 105 of housing 102, and display 112 is located at second end 107 of housing 102. In certain embodiments, the display can be directly attached to the top of the housing. For example, in FIG. 1, display 112 is directly attached to the top of housing 102.

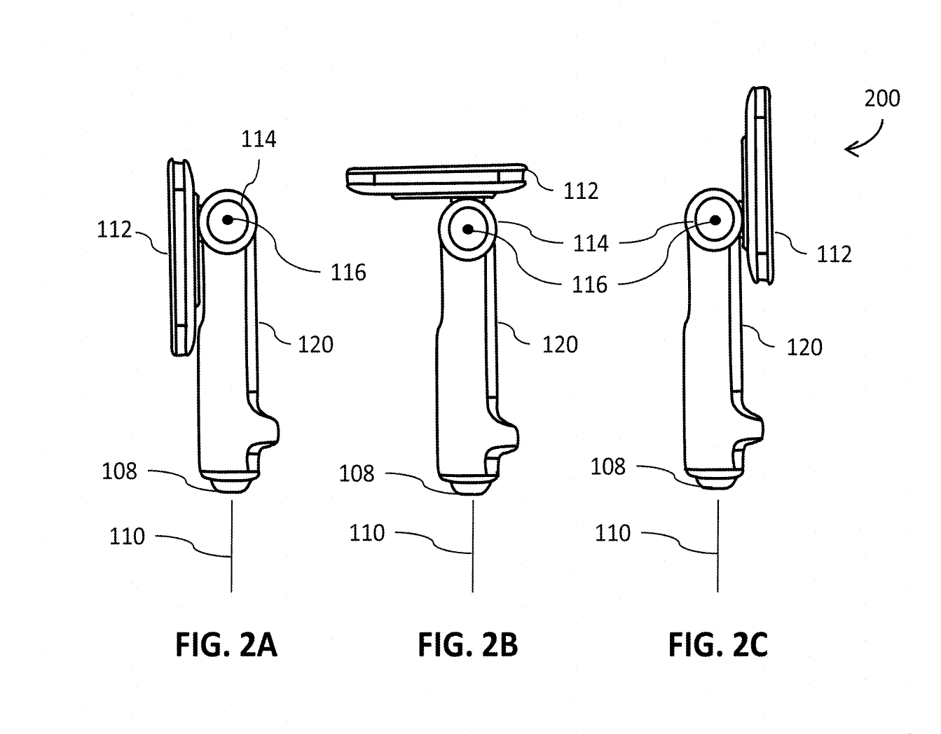

According to certain embodiments, the display of the imaging unit can be a rotatable display. For example, referring to FIG. 1, display 112 can be connected to housing 120 via pivot 114, which can allow rotatable display 112 to rotate about at least one rotational axis 116. Any suitable pivot may be used. For example, as shown in FIG. 1, cylindrical pivot 114 can be used to rotate display 112 about a single rotational axis 116. FIGS. 2A-2C are exemplary schematic illustrations showing the rotation of display 112 about single rotational axis 116 (going into and out of the page in FIGS. 2A-2C). In FIGS. 2A-2C, display 112 is rotated about device handgrip region 120. In certain embodiments, pivot 114 comprises a ball-and-socket arrangement or other type of arrangement, which can allow for rotation of display 112 about multiple rotational axes. In some such embodiments, display 112 can be rotated about more than one axis so that twisting or rotation in other dimensions is allowed. In other such embodiments, the display 112 can be attached to the housing via magnets, hook and loop fastener (e.g., Velcro), or other attachment means.

In some embodiments, the rotatable display is configured to be rotated, relative to at least another portion of the housing (e.g., the handgrip region, the imaging unit, and/or another portion of the housing), about at least one rotational axis by at least about 30 degrees, at least about 60 degrees, at least about 90 degrees, at least about 120 degrees, at least about 150 degrees, or at least about 175 degrees. For example, in FIGS. 2A-2C, display 112 is illustrated as being rotated about both handgrip region 120 and ultrasound transducer 108 by 180 degrees.

In certain embodiments, the rotatable display is configured to be rotated such that the display can be oriented in a first position substantially parallel to a scanning plane of the imaging unit and in a second position substantially perpendicular to the scanning plane of the imaging unit. For example, in FIGS. 2A and 2C, imaging device 100 is shown in which display 112 is substantially parallel to scanning plane 110 of imaging device 100. In addition, in FIG. 2B, display 112 is substantially perpendicular to scanning plane 110 of imaging device 100. Display 112 can be said to be in a 0-degree configuration in FIG. 2A, a 90-degree configuration in FIG. 2B, and a 180-degree configuration in FIG. 2C. In the exemplary embodiment of FIGS. 2A-2C, display 112 can be configured to a position at any angle relative to scan plane 110 of imaging device 100 within the range between the 0-degree configuration (FIG. 2A) and the 180-degree configuration (FIG. 2C).

While a rotatable display has been described in association with the above embodiments, it should be understood that the invention is not so limited, and in other embodiments, non-rotatable displays could be used.

In certain embodiments, the housing is elongated and comprises a longitudinal axis. For example, referring back to FIG. 1, housing 102 is elongated and comprises longitudinal axis 106.

In some embodiments, the imaging device is configured such that the smallest angle between the scanning plane 110 of the ultrasound imaging unit 104 and the longitudinal axis 106 of the device housing 102 is relatively small (e.g., less than about 45°, less than about 30°, less than about 15°, less than about 5°). In certain embodiments, the smallest angle between the scanning plane 110 of the ultrasound imaging unit 104 and the longitudinal axis 106 of the device housing 102 is less than about 1°, in which case, the scanning plane 110 of the ultrasound imaging unit 104 is said to be substantially parallel to the longitudinal axis 106 of the device housing 102.

In some embodiments, the imaging device can be relatively small. For example, in certain embodiments, the imaging device may occupy a volume of equal to or less than about 500 cm³ or less than about 100 cm³ (as determined by sealing any openings on the external surfaces of the imaging device and measuring the volume of liquid displaced by the sealed imaging device). In some embodiments, the imaging device is configured such that the entire device may be arranged to fit into a pocket. For example, the ultrasound imaging device can be configured such that the entire device fits into a side pants pocket, according to certain embodiments.

In some such embodiments, the imaging device is portable such that it may be, for example, carried or otherwise manipulated by hand (e.g., by a single hand). In some embodiments, the imaging device is completely self-contained with portable dimensions such that it can be manipulated with one hand.

In certain embodiments, the housing comprises a hand grip region (e.g., a handle), which can be configured to be grasped by the user during operation of the imaging device. In some embodiments, the hand grip region can be between the ultrasound imaging unit and the display. For example, referring to FIG. 1, hand grip region 120 of imaging device 100 is positioned between imaging unit 104 and display 112. In certain embodiments, the hand grip region may be between the imaging interface of the ultrasound transducer and the display. The imaging interface of an ultrasound transducer is known to those of ordinary skill in the art to refer to the interface of the transducer from which the ultrasonic signal emanates. For example, referring to FIG. 1, ultrasound transducer 108 comprises imaging interface 109.

The hand grip region (e.g., handle) may be positioned, in certain embodiments, such that it is directly above the imaging unit during operation of the imaging device. For example, referring to FIG. 1, hand grip region 120 is positioned such that it is directly above imaging unit 104 (as well as transducer 108 and imaging interface 109 of transducer 108) during operation of imaging device 100.

In some embodiments, the imaging device can be battery operated. For example, in certain embodiments, the imaging device is powered using a cell-phone class battery, such as a 2000 mAh Li-ion battery.

In certain embodiments the display can be integrated with the device housing (e.g., such that display and the device housing form a monolithic unit). For example, referring to FIG. 1, device housing 102 is integrated with imaging unit 104 (and transducer 108) to form an integrated, monolithic unit. In addition, in FIG. 1, display 112 and device housing 102 are integrated to form a monolithic unit. In other embodiments the display can be detachable from the housing unit. In some such embodiments, the ultrasound imaging unit (e.g., 104 in FIG. 1) can communicate with the display via wireless connection.

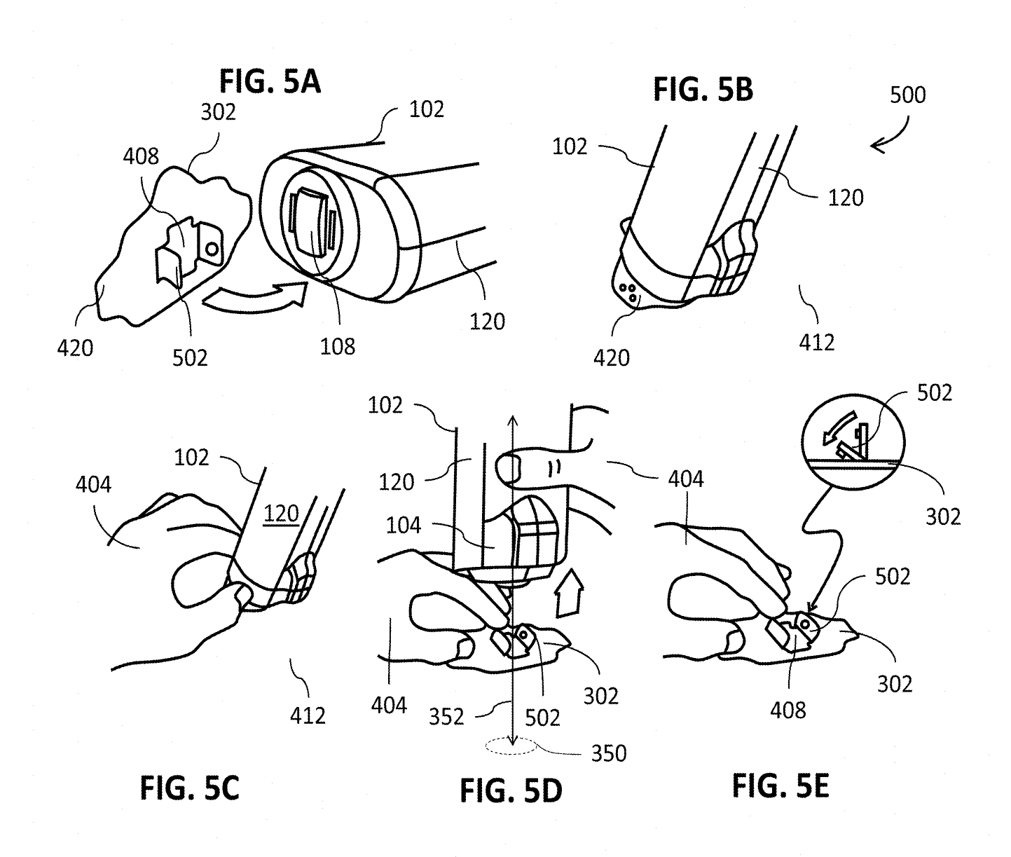

Certain of the imaging devices described herein can provide one or more of a variety of advantages, relative to prior devices. For example, according to certain embodiments, by attaching the display to the base of the housing (e.g., in direct contact with the base of the housing), the display can be located relatively close to the target site that is being imaged (e.g., a subject such as a human subject). This can make, according to certain embodiments, operation of the inventive imaging device easier than systems that include, for example, a transducer probe with a cable and the display that is not in the same vicinity as the user's hand and probe. In certain embodiments, the closest distance between the image display and the target site (e.g., skin surface 412 in FIGS. 4A-4D) is less than about 1 meter, less than about 500 cm, less than about 100 cm, less than about 50 cm, or less than about 25 cm.

Certain of the embodiments described herein do not include a separate transducer-to-display cable, which would otherwise complicate operation and handling of the imaging device. In addition, as noted above, the imaging device can be configured to be portable and adopt a form factor that can be more easily transported in a user's pocket. While other handheld ultrasound devices have been proposed with an attached screen (i.e., no transducer-to-display cable), such systems possess a form factor where the hand is placed along a handle region that is extended from the transducer face. That is to say, in such systems, the longitudinal axis of the handle forms a smallest angle with the imaging plane of the imaging unit that is close to 90 degrees. In contrast, certain of the embodiments described herein place the hand immediately above the transducer face, which possesses the benefit of allowing for finer control of the scan plane. That is to say, certain of the embodiments described herein include a smallest angle between the longitudinal axis of the hand grip region and the imaging plane of the imaging unit that is relatively small (e.g., a smallest angle of less than about 45°, less than about 30°, less than about 15°, or less than about 5°) and/or are configured such that the longitudinal axis of the hand grip region is substantially parallel to the imaging plane of the imaging unit. Those of ordinary skill in the art are familiar with the longitudinal axis of a hand grip region, which refers to the elongated axis around which the hand grasps when grasping the handgrip region. In certain embodiments, the longitudinal axis of the handgrip region corresponds to the longitudinal axis of the housing in which the handgrip region is formed.

According to certain embodiments, there may be a clinical benefit to using an ultrasound imaging device in which the display screen is housed within the same device housing as the imaging unit and/or is rotatable about the longitudinal axis of the device. In some embodiments, the rotating display can allow the user to adjust the display angle to account for different angles between the user's eye level and the central longitudinal axis of the device. Thus, the user may hold the imaging device above, below, to the left of, or to the right of their eye level and still have a good viewing angle to the display. The ability to adjust the eye level can be important for scanning different anatomies with the same device while still being able to view the display screen. As one example, performing a cardiac scan and a lumbar spine scan while maintaining the user's body in a substantially fixed position (e.g., standing or sitting) will generally necessitate positioning the imaging device such that the angle between the device and the user's eye level varies. Without a rotatable screen, the user would need to substantially move their body orientation in order to view the screen with sufficient clarity for both types of scans. With the rotatable screen, however, there is greater flexibility in uses of the ultrasound device, while maintaining a smaller form factor compared with ultrasound devices that have a separate transducer and display screen connected by a cable.

Moreover, positioning the hand grip region as illustrated in FIG. 1 (and other figures) can produce greater control over the position of the scan plane and/or can facilitate clinical acceptance. Precise control over the scan plane relative to the anatomy of interest is often important in ensuring that the anatomy is accurately captured by the scan plane and can be assessed via review of the display screen. In certain embodiments in which the hand grip region is configured such that the longitudinal axis of the hand grip region is substantially parallel to the imaging plane, the user's hand can be used to control the imaging device by placing the hand on the hand grip region while also resting the hand on the target skin surface. During testing of this configuration, it was discovered that arranging the imaging unit components in this manner leads to greater stability. Without wishing to be bound by any particular theory, it is believed that this increase in stability is observed because the hand, target skin surface, and device are all connected and can all move together. Frequently, a subject (e.g., a patient) may move during an ultrasound scan (e.g., due to discomfort, restlessness, or for another reason). Certain embodiments, including that illustrated in FIG. 1 and other figures can allow for a stable image to be captured regardless of patient motion.

In certain embodiments, the imaging device comprises an actuator on the hand grip region configured such that when the actuator is activated, data from the ultrasound imaging unit is recorded and/or manipulated. For example, in FIG. 1, imaging device 100 comprises actuator 130, which is in the form of a button.

According to certain embodiments, when actuator 130 is depressed, data from the ultrasound imaging unit is recorded, for example, to memory within the ultrasound imaging device or to an external memory unit outside the imaging device (e.g., after being transported via a wireless or wired connection between the imaging unit and the external memory unit). The data from the ultrasound imaging unit that is recorded can correspond to, for example, imaging data (e.g., data related to a B-mode image, a C-mode image, an M-mode image, a tissue harmonic image, a three-dimensional image, a Color Doppler image, a Power Doppler image, a Pulse-wave Doppler image, a continuous wave Doppler image, an ultrasound contrast agent enhanced image, a B-flow image, or any other type of image). In this way, the actuator can be configured to take a “snapshot” of the data being collected by the ultrasound imaging device at that point in time during its use. In some embodiments, the actuator can be configured to perform one or more of a number of other functions including: video save, device power on/off, image settings adjustment (e.g., imaging mode, gain, frequency, contrast, depth), and/or menu navigation. In some embodiments, the imaging device 100 comprises a plurality of actuators, such as the actuator 130 in FIG. 1, which can perform different functions, such as the aforementioned functions.

While the actuator illustrated in FIG. 1 is a button, any suitable actuator type can be used. Examples include, but are not limited to, touch sensors (e.g., resistive, capacitive, optical), switches, proximity sensors, and/or optical sensors.

It can be advantageous, according to certain but not necessarily all embodiments, to locate the actuator on the hand grip region of the imaging device. Locating the actuator on the hand grip region can allow a user to activate the data saving function, or other aforementioned functions, relatively easily, for example, by simply activating the actuator using a digit of the hand (e.g., a finger and/or thumb) holding the imaging device. In some embodiments, the hand may activate the actuator for purposes of activating a desired function, such as a saving function, simultaneously while performing an image scan without requiring the user to reorient the hand on the hand grip region of the ultrasound device. That is to say, according to certain embodiments, the user does not need to reorient their hand to save an image during an ultrasound scan.

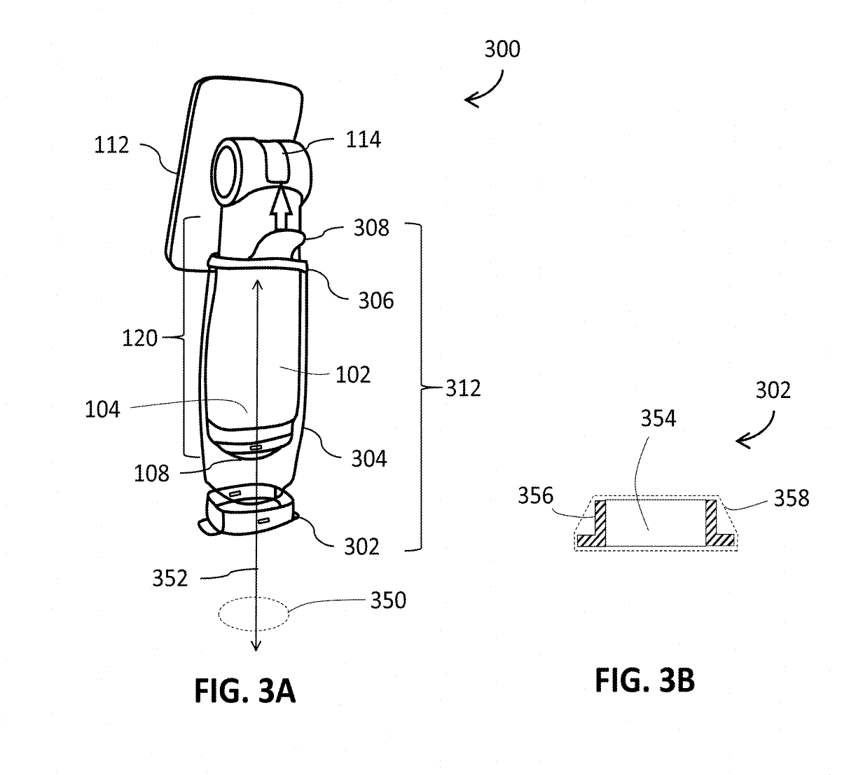

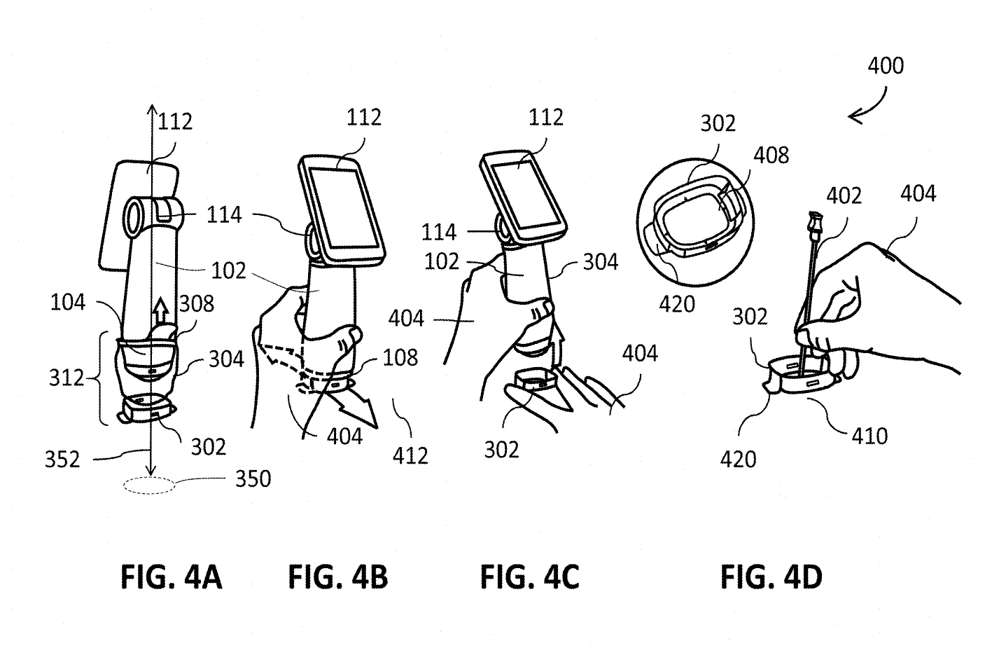

According to certain embodiments, the imaging device comprises a marking unit detachably coupled to the housing. For example, in FIG. 3A, imaging unit 300 comprises marking unit 302, which can be detachably coupled to housing 102. Similar arrangements are illustrated in the embodiments shown in FIGS. 4A-4D, 5A-5E, 6, and 7, described in more detail below.

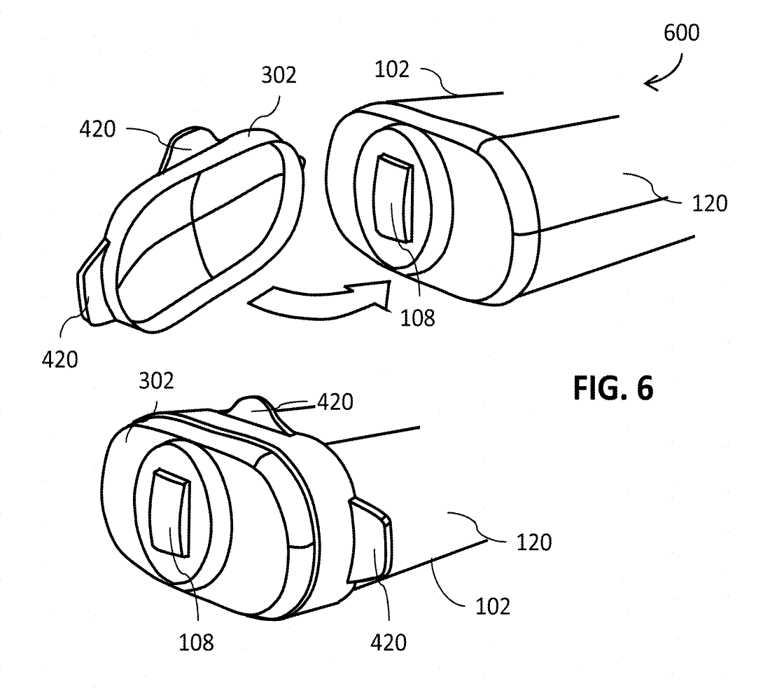

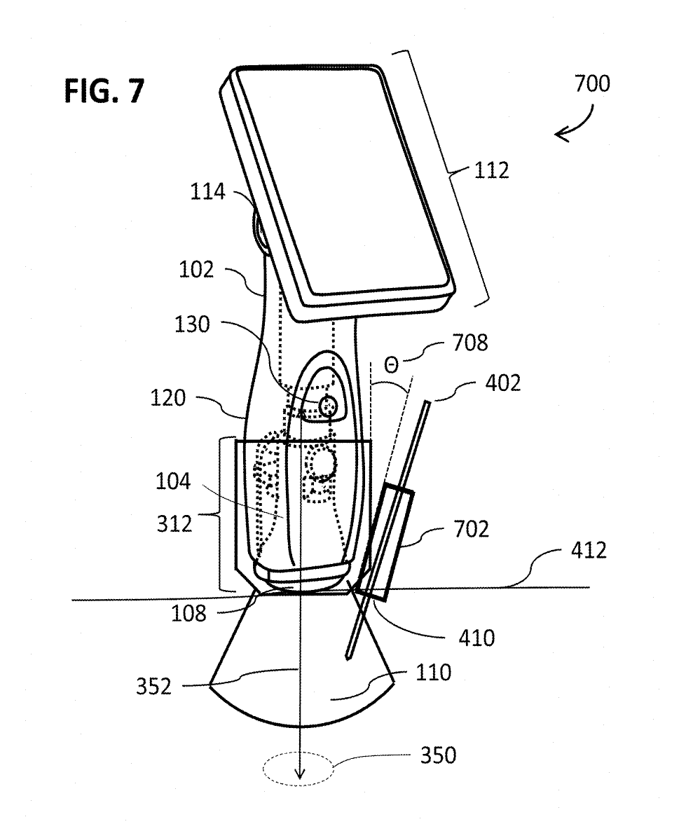

The marking unit and the housing can be detachably coupled in any suitable fashion. In some embodiments, the marking unit can be indirectly coupled to the housing. For example, as described in more detail below, according to certain embodiments, a cover body is attached to the housing. In some such embodiments, the marking unit is detachably coupled to the cover body. Exemplary embodiments of such an arrangement are illustrated in FIGS. 3A and 4A-4D. In certain embodiments, the marking unit is present alone, without the cover body. In some such embodiments, the marking unit is directly coupled to the housing. For example, in FIGS. 5A-5E and 6, marking unit 302 is directly coupled to the housing of the imaging unit. The marking unit can be “self-attaching”to the imaging device (or the cover body, when present), in certain embodiments. That is to say, in some embodiments, substantially no separate attaching material (such as a rubber band) is required to attach the marking unit to the imaging device (or to the cover body, when present). Examples of mechanisms that can be used to attach the marking unit to the imaging device (and/or the cover body, when employed) in this way include, but are not limited to, magnetic fittings (e.g., a pair of magnets between the housing and the marking unit), mechanical attaching mechanisms (such as a fitted plastic inserts, spring-loaded retaining clips, frictional fittings, elastic bands, hook and loop fasteners, screw threads), and the like. Of course, other attachment mechanisms, such as adhesive-based attachment mechanisms, could also be used.

The marking unit can be configured to indicate proper placement of a probe (e.g., a needle and/or a catheter) along a target that is to be imaged, in some embodiments. In certain embodiments, the marking unit is configured to identify a target surface location (e.g., an insertion location) corresponding to a center of an imaging scan plane. The marking unit can comprise, in certain embodiments, a probe indicator configured to indicate proper placement of a probe at or near a target that is to be imaged. For example, in some embodiments, the marking unit comprises an identifying mark indicating the target surface location. The identifying mark can comprise, for example, a hole, an indentation, or other identifying mark.

In some such embodiments, the detachable marking unit can be mechanically removed from the imaging device during use. In some such embodiments, the detachable marking unit 302 can be left at or near the target site (e.g., on the skin) to identify the center of the transducer along the skin after the transducer itself has been removed.

FIGS. 4A-4D are exemplary schematic illustrations outlining such use of the detachable marking unit. In FIG. 4A, the detachable marking unit is used to identify the location of needle insertion (i.e. the target insertion location 410) in a spinal anesthesia procedure, although use of the detachable marking unit to locate other types of target anatomy (e.g., blood vessels, nerves, joints, or organ tissues) is also possible. In FIG. 4A, marking unit 302 is placed over an imaging device hand grip region 120. Marking unit 302 can be configured to indicate proper placement of a needle (shown as 402 in FIG. 4D) along a target skin surface 412, which is to be the imaging surface, in some embodiments.

As shown in FIG. 4B, ultrasound imaging device 100 can be scanned across target skin surface 412 with a user's hand 404 until the user identifies the target anatomy in the ultrasound image displayed on device display 112. According to certain embodiments, marking unit 302 can be configured to identify a location along skin surface 412 that corresponds to the center of the scanning plane produced by ultrasound transducer 108 of the imaging device.

Next, as illustrated in FIG. 4C, marking unit 302 can be detached by user's hand 404 from both the device hand grip region 120 (and optional cover body 304, described in more detail below). In certain embodiments, the marking unit is configured to be attached to a target surface. For example, in some embodiments, the marking unit comprises an adhesive material configured to adhere the marking unit to a surface of a target (e.g., the skin of a subject). In FIG. 4C, for example, marking unit 302 can comprise an adhesive material configured to adhere marking unit 302 to skin surface 412. Other attachment mechanisms could also be employed. For example, in some embodiments, the marking unit comprises a vacuum-generating unit configured to adhere the marking unit to a surface associated with the target. The marking unit 302 can comprise, for example, suction cups or other vacuum means to support adhesion of the marking unit to the target surface.

As noted above, in certain embodiments, the marking unit comprises, a probe indicator configured to indicate proper placement of a probe at or near a target that is to be imaged. For example, in some embodiments, the marking unit comprises an identifying mark indicating the target surface location. The identifying mark can comprise, for example, a hole, an indentation, or other identifying mark. In FIG. 4D, marking unit 302 comprises identifying marks 408, which are configured to indicate the proper placement of needle 402 along skin surface 412. In certain embodiments, the marking unit 302 comprises a hole 408 in the middle where the center of the transducer 108 was located when the unit 302 was detached from the imaging device housing, such that when left on skin 412, marking unit 302 identifies the place along skin surface 412 corresponding to the center of ultrasound scan plane 110.

According to certain embodiments, with the marking unit 302 adhered to the skin surface 412, the user can insert a probe 402 at the target insertion location 410 using the hole guide 408 along the marking unit 302.

The detachable marking unit, in some embodiments, can comprise a wing component. For example, referring to FIG. 4D, detachable marking unit 302 comprises wing 420. The wing component can be advantageous, according to certain although not necessarily all embodiments, because it can improve one's ability to adhere the marking unit to a target skin surface by increasing surface area of the marking unit along the target skin surface.

While identifying marks are illustrated in FIG. 4D as guiding the placement of the probe, other embodiments are also possible. For example, in some embodiments, the marking unit comprises a moveable tab configured to indicate a site corresponding to a center of a scan plane. The moveable tab can be configured, in certain embodiments, to make a visible indentation at a site corresponding to the center of the scan plane (which can correspond, for example, to the center of a cavity, hole, or other indicator formed in the marking unit). FIGS. 5A-5E is a set of schematic illustrations outlining the use of a moveable tab to indicate a target region. In FIG. 5A, tab 502 can be folded into a center cavity 408 within the marking unit 302 to make an indentation along target skin surface 412 at target insertion location 410 corresponding to the center of cavity 408. In FIG. 5A ultrasound transducer 108 is illustrated with an exemplary embodiment of the marking unit 302 comprising a cavity 408 and moveable tabs 502. Marking unit 302 can be attached to the hand grip region 120 of the ultrasound device 100 to cover transducer region 302. FIG. 5B illustrates ultrasound device 100 with marking unit 302 attached and pressed against the target skin surface 412 where ultrasound imaging can be conducted. As shown in FIG. 5C, a user's hand 404 can press against marking unit tab regions 420 in order to remove marking unit 302 from ultrasound device 100 while maintaining marking unit 302 along the target skin surface 412 at the target insertion location 410. As illustrated in FIG. 5D, marking unit 302 can be removed from imaging device 100. As shown in FIG. 5E, the user's hand 404 can activate moveable tabs 502, which can make an indentation along target skin surface 412 at the target insertion location 410. According to certain embodiments, a probe may then be inserted at the point at which the indentation has been made.

FIG. 6 is a schematic illustration showing an exemplary mechanical attachment scheme that can be used to connect a marking unit 302 with an imaging device 100. The marking unit 302 in FIG. 6 is configured to “self-attach” to the device housing 102. Self-attachment can be achieved by previously described means including, in one embodiment, a form fitting between the device housing 102 and marking unit 302 such that frictional forces are sufficient to ensure attachment. The wings 420 of the marking unit 302 in FIG. 6 can be advantageous, according to certain although not necessarily all embodiments, because it can improve one's ability to adhere the marking unit to a target skin surface. Additionally, wing regions can provide areas whereby a user can press against to detach the marking unit 302 from the device housing 102.

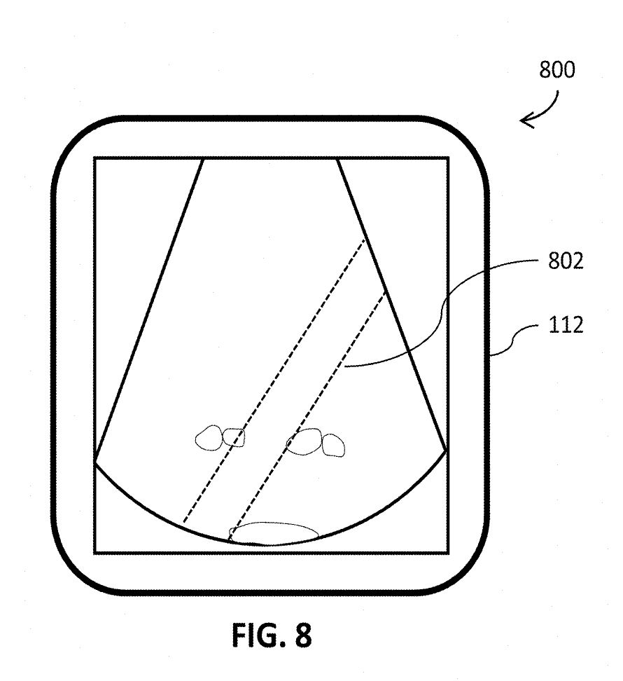

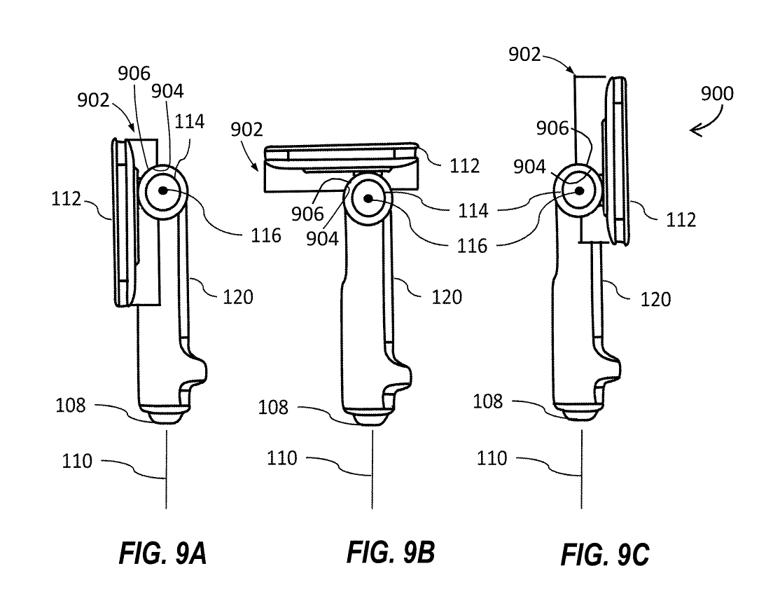

In some embodiments, the marking unit 302 comprises a sleeve 702 comprising an elongated lumen passing through it. The sleeve can be used as a probe indicator, as described above. For example, the elongated lumen of the sleeve can be used to guide or otherwise house an elongated device such as a probe 402. FIG. 7 is a schematic illustration of one set of embodiments in which a cover 312 comprises a sleeve 702, which is being used to house a probe, such as a needle 402. The sleeve 702 can be configured to control the angle 708 of the elongated probe 402 that is threaded through the lumen (e.g., a needle 402 to be inserted at the target region 350). The imaging device display 112 can convey the angle 708 information with indicators such as, for example, dashed lines 802 superimposed on the ultrasound image. The user can then manually scan the imaging device 100 across the target skin surface 412 until the dashed line(s) 802 pass through the target of interest (e.g., blood vessel, nerve structure, epidural space) and then project the probe 402 through the sleeve 702. An example image display 112 in which overlaying indicator lines 802 are used to indicate the projected path of a probe 402 relative to the image if inserted through the sleeve 702 is illustrated in FIG. 8.

As noted above, certain aspects are related to configurations of a handheld ultrasound imaging device in which the target that is being imaged, the ultrasound imaging unit, and the marking unit are aligned during use. Alignment of these components during use can make location of target regions relatively easy for the user. According to certain embodiments, the marking unit is positioned such that, during use, a line extending between the target and the ultrasound imaging unit intersects the marking unit.

For example, referring to FIG. 3A, target region 350, marking unit 302, and imaging unit 104 are aligned such that line 352 (which extends infinitely, as indicated by the arrow at the ends of the line) intersects marking unit 302. It should be understood that a line is said to “intersect” a marking unit when the line passes through any region that lies within the outer geometric boundary of the marking unit, which includes both regions of the solid material that makes up the marking unit as well as voids (e.g., holes and the like) within such regions. As an illustrative example, FIG. 3B is a schematic cross-sectional schematic illustration of marking unit 302 shown in FIG. 3A. Marking unit 302 includes cavity 354 formed within a surrounding solid material 356, and defines outer geometric boundary 358. Although cavity 354 is not formed of any material per se, a line extending through cavity 354 would still be said to “intersect” marking unit 302.

Referring to FIGS. 4A-4C, marking unit 302, imaging unit 104, and target region 350 are also aligned such that line 352 intersects marking unit 302. In FIG. 5D, marking unit 302, imaging unit 104, and target region 350 are aligned such that line 352 intersects marking unit 302. In FIG. 7, imaging unit 104, and target region 350 are aligned such that line 352 intersects marking unit 302.

According to certain embodiments, the longitudinal axis of the housing intersects the marking unit (and, in some such embodiments, the longitudinal axis of the housing extends between the target and the ultrasound imaging unit). In some embodiments, the longitudinal axis of the hand grip region intersects the marking unit (and, in some such embodiments, the longitudinal axis of the hand grip region extends between the target and the ultrasound imaging unit).

According to certain embodiments, during use, the marking unit is positioned between the ultrasound imaging unit and the target that is to be imaged. For example, in FIG. 3A, marking unit 302 is positioned such that imaging unit 104 is above marking unit 302, and target region 350 is below marking unit 302. In other embodiments, the imaging unit could be located between the marking unit and the target region, for example, by sliding the marking unit up the hand grip region 120 of the housing.

In certain embodiments, a cover for an ultrasound imaging device is described. The cover comprises, in certain embodiments, a cover body configured to be attached to an imaging device, and a marking unit attached to the cover body and configured to indicate proper placement of a probe (e.g., a needle and/or a catheter) along a target that is to be imaged. Such imaging can allow a target anatomy to be reached with the probe, according to certain embodiments.

FIG. 3A is an exemplary schematic illustration of a cover 312 including a cover body 304 and detachable marking unit 302 connected to an imaging device 100. In FIG. 3A, the cover body covers the hand grip region 120 (e.g., including the imaging unit 104 and ultrasound transducer 108). The cover 312 can be used, in certain embodiments, as a sterile shield to the target skin surface 412 (e.g., the skin of a subject). According to certain embodiments, cover 312 can comprise an elastomeric band 306 and tab 308, for example, to facilitate the placement of cover 312 over hand grip region 120.

The cover body can be “self-attaching” to the imaging device, in certain embodiments. That is to say, in some embodiments, substantially no separate attaching material (such as a rubber band) is required to attach the cover body to the imaging device. Examples of mechanisms that can be used to attach the cover body to the imaging device in this way include, but are not limited to, a pair of magnets between the cover body and the hand grip region, mechanical attaching mechanisms (such as a fitted plastic inserts, spring-loaded retaining clips, elastic bands), and the like.

In certain embodiments, cover body 304 and/or marking unit 302 may be disposable. In some embodiments, cover body 304 and/or marking unit 302 may be sterile.

In certain embodiments in which a cover body is employed, the cover body and the marking unit can be removably attached to each other. For example, the cover body and the marking unit can be attached to each other, in certain embodiments, such that separation of the cover body and the marking unit does not damage either of the cover body and the marking unit. In some embodiments, the cover body and the marking unit can be attached to each other such that separation of the cover body and the marking unit can be achieved by hand, and does not require an additional tool (e.g., a screwdriver or other such tool). Examples of mechanisms that can be used to attach the marking unit to the imaging device (and/or the cover body, when employed) in this way include, but are not limited to, magnetic fittings (e.g., a pair of magnets between the housing and the marking unit), mechanical attaching mechanisms (such as a fitted plastic inserts, spring-loaded retaining clips, frictional fittings, elastic bands, hook and loop fasteners, screw threads), and the like.

In some embodiments, the marking unit is configured such that when it is attached to the imaging device, an image can be produced without the use of transmission medium. According to certain embodiments, when marking unit is attached to the imaging device, an image can be produced without substantial acoustic attenuation derived from the marking unit and without the use of transmission medium. For example, in some embodiments, less than 6 dB round-trip acoustic attenuation is observed at 1 MHz center frequency, without the use of transmission medium, when the marking unit is positioned as described elsewhere herein (e.g., when the marking unit is aligned with the ultrasound imaging unit and the target, as described elsewhere herein). In some embodiments, the cover can be configured such that when it is attached to an ultrasound imaging device, received ultrasound data can be recorded for display of an image to the device display without the use of ultrasound gel or other ultrasound transmission medium being placed in between the transducer and the cover and/or marking unit. In contrast, other ultrasound imaging systems generally require the use of ultrasound gel to produce an image with acceptable image quality. The requirement for ultrasound transmission medium between the transducer and cover and/or marking unit can be eliminated, according to certain embodiments, by attaching the cover and/or marking unit to the transducer such that substantially no air is present between the two objects. For example, in some embodiments, an elastomeric band or other attaching mechanism can be configured such that the when the cover is attached to the ultrasound imaging device, it is pressed with high tension against the transducer surface. The tension can be made to be sufficiently high such that there substantially no air can be present between the two objects. In this way, the ultrasound beam can transmit between the two materials (i.e. transducer and cover) without requiring transmission medium. Exemplary materials from which the marking unit can be made include, for example, polyurethane, polyethylene, and silicone.

In some embodiments in which a cover body is employed (in addition to the marking unit), the cover is configured such that when it is attached to the imaging device, an image can be produced without substantial acoustic attenuation derived from the cover and without the use of transmission medium. For example, the cover can be configured, in certain embodiments, such that when it is attached to an ultrasound imaging device, received ultrasound data can be recorded for display of an image to the device display without the use of ultrasound gel or other ultrasound transmission medium being placed in between the transducer and the cover. The cover can be configured for us without transmission medium, for example, by using a cover material that is thin (e.g., less than about 5 mm thick) and/or by employing a self-attaching mechanism that holds the cover material against the transducer face with sufficient strength during operation of the imaging device. In this way, substantially no air is trapped between the cover and transducer face, and the cover itself is too thin to cause appreciable attenuation or reflections that can lead to unacceptable image quality (e.g., less than 6 dB round-trip acoustic attenuation at 1 MHz center frequency). Example cover materials can include, for example, polyurethane, polyethylene, and silicone.

As noted above, the ultrasound imaging devices described herein can produce ultrasound images using a variety of known techniques. In certain embodiments the ultrasound transducer (e.g., ultrasound transducer 108) can include a mechanically scanned single element transducer. In some embodiments, the ultrasound transducer can be a linear array, a two-dimensional array, or an annual array. In certain embodiments the ultrasound imaging unit (e.g., ultrasound imaging unit 104) can be configured to produce, for example, a B-mode image, a C-mode image, an M-mode image, a tissue harmonic image, a three-dimensional image, a Color Doppler image, a Power Doppler image, a Pulse-wave Doppler image, a continuous wave Doppler image, an ultrasound contrast agent enhanced image, a B-flow image, or any other mode or combination of modes whereby an image is formed from information received by the ultrasound transducer. Those of ordinary skill in the art of ultrasound understand that an ultrasound imaging unit generally comprises a combination of one or more of an ultrasound transducer, and circuitry and processing units for conditioning, processing, and transferring image data to the display unit. The ultrasound imaging unit can be contained within the device housing. For example, in some embodiments, the ultrasound-imaging unit can comprise an ultrasound transducer, an ultrasonic signal conditioning circuit, and a processor circuit, which can be communicatively connected via a bus. The ultrasonic signal conditioning circuit can include a number of conventional processing circuitries such as beam-forming circuitry or other processing circuitry. For example, the ultrasonic signal conditioning circuit can be configured to amplify, phase-shift, time-gate, filter, or otherwise condition received ultrasonic information (e.g., echo information), such as provided to the processor circuit. In a further example, the receive path from each transducer element can include one or more of a low noise amplifier, a main-stage amplifier, a band-pass or a low-pass filter, or an analog-to-digital converter. In one example, one or more signal conditioning steps can be performed digitally, such as by using the processor circuit. The term processor is used to generically refer to digital circuitry that can be used to manipulate ultrasound information obtained from the ultrasound transducer. Such circuitry can include one or more of a field-programmable gate array (FPGA) or other programmable logic devices (PLDs), microprocessor, a system-on-chip including one or more execution cores or other circuitry, a microcontroller, or one or more or other circuits. Those of ordinary skill in the art of ultrasound and image processing will understand that the signal conditioning and processing steps and their order of operation to be performed by the signal conditional circuit and processor circuit will vary depending on the desired image to be rendered to the display (e.g., B-mode image, C-mode image, M-mode image, tissue harmonic image, three-dimensional image, Color Doppler image, Power Doppler image, Pulse-wave Doppler image, continuous wave Doppler image, ultrasound contrast agent enhanced image, or B-flow image).

U.S. Provisional Patent Application Ser. No. 61/770,448, filed Feb. 28, 2013, and entitled “Localization of Imaging Target Regions and Associated Systems and Devices,” is incorporated herein by reference in its entirety for all purposes.

As stated above, many ultrasound procedures require that ultrasound scanning be performed in a sterile manner. This is especially true for ultrasound guidance of needle or catheter placements or intraoperative ultrasound. Current ultrasound systems are made sterile by placing a sterile sleeve over the ultrasound transducer. This currently requires two people. A first person, who is wearing sterile gloves, holds a sterile sleeve, while a second person places a non-sterile ultrasound transducer into the sterile sleeve. In the course of such process, the non-sterile ultrasound transducer and the person holding it must not touch the person wearing sterile gloves.

However, it has been determined that a handheld ultrasound-imaging device may be provided with a configuration that allows one person to apply a sterile sleeve or other type of sterile cover over the ultrasound transducer, at least in part, without help from another person.

In at least some embodiments, such a configuration and method is beneficial and less time and resource consuming than current systems and methods.

In that regard, in at least some embodiments, a handheld ultrasound-imaging device may have a housing (for an ultrasound transducer), a rotatable display and a configuration that allows the rotatable-display (a housing thereof or otherwise) to be used as a stand, and further allows the ultrasound transducer to be positioned at least partly upright, with a membrane of the ultrasound transducer facing up. The device further includes a brace to resist (to at least some degree) movement of the housing relative to the rotatable-display.

With such configuration, the handheld ultrasound-imaging device may be positioned on a surface (non-sterile or otherwise), a gel or other ultrasound transmission medium may be applied to the ultrasound transducer without falling off, and a person wearing sterile gloves may apply a sterile cover on the ultrasound transducer, at least in part, while touching only the sterile cover.

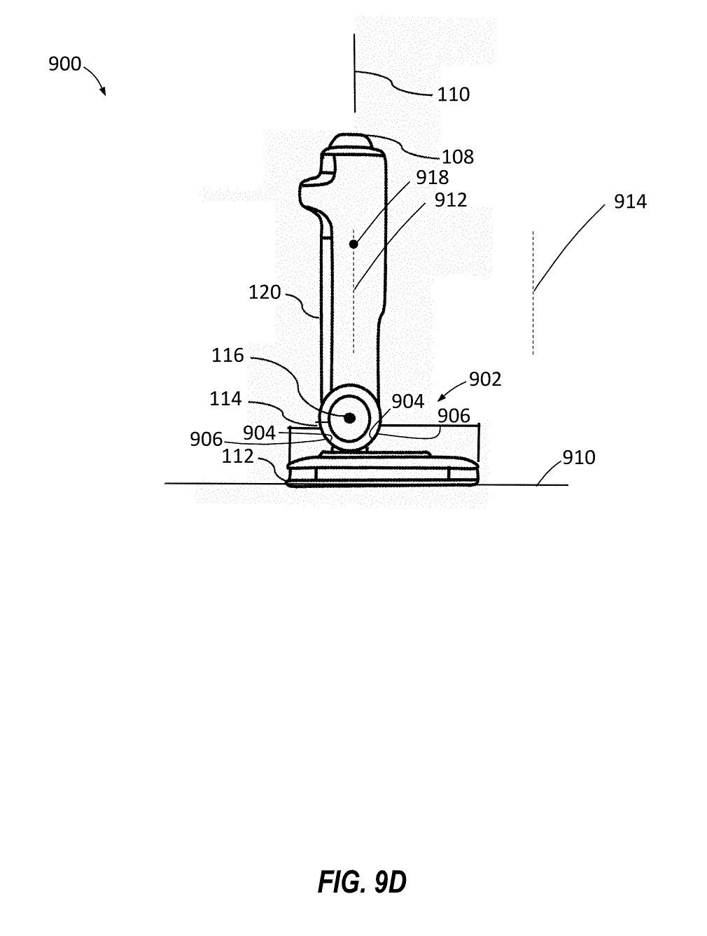

FIGS. 9A-9D show schematic illustrations of a handheld ultrasound-imaging device 900 that is similar to one or more embodiments of the handheld ultrasound-imaging device 100 disclosed above, except that the handheld ultrasound-imaging device 900 includes a brace 902, in accordance with at least some embodiments.

Referring to FIGS. 9A-9D, the brace 902 may include a brace surface 904 that is external to said pivot 114 and in contact at least in part (a major part or a minor part) with a surface 906, that is complementary to the brace surface 904 or otherwise, and that moves relative to said brace surface 904 with relative movement between the housing 102 and the display 112 to thereby provide a friction force that resists rotation of the housing 102 relative to the display 112. In at least some embodiments, the housing 102 may define the surface 906. In at least some embodiments, the pivot 114 may define the surface 906. In at least some such embodiments, the pivot 114 may comprise an assembly (which may include a housing) having an external surface that defines the surface 906.

FIG. 9D shows the handheld ultrasound-imaging device 900 with the display 112 (a housing thereof or otherwise) being used as a stand (and at rest on a horizontal surface 910), the housing 102 in an upright position relative to the display 112, and the brace resisting (to at least some degree) movement of the housing 102 relative to the display 102.

Unless stated otherwise, the term “upright position” means a fully upright position and/or a partly upright position.

In at least some embodiments, including but not limited to the illustrated embodiment, the housing 102 may have a longitudinal axis, e.g., longitudinal axis 912, which may be parallel to a vertically axis 914 when the display 112 on a horizontal surface, e.g., horizontal surface 910, and the housing 102 is in a fully upright position.

Although the housing 102 is shown in a fully upright position, in at least some embodiments, partially upright positions may also be employed.

In at least some embodiments, it may be desirable to position the housing such that its center of gravity, e.g., center of gravity 918, is disposed directly above the pivot 114 and/or a portion thereof. In at least some embodiments, it may be desirable to position the housing 102 such that its center of gravity, e.g., center of gravity 918, is disposed directly above the axis 116 and/or a portion thereof.

In at accordance with at least some embodiments, including but not limited to the illustrated embodiment, when the display 112 is used as a stand and at rest on surface, e.g., horizontal surface 910, the screen of the display 112 faces toward such surface.

Unless stated otherwise, the phrase “face toward” means “face toward”, to at least some degree and/or at least some extent.”

Unless stated otherwise, the phrase “face away” means “face away”, to at least some degree and/or at least some extent.”

The brace 902 may have any configuration (e.g., size, shape, etc.) and/or positioning.

In at least some embodiments, including but not limited to the illustrated embodiment, the brace surface 904 may comprise a concave surface and the surface 906 may comprise a convex surface.

In at least some embodiments, including but not limited to the illustrated embodiment, the brace 902 may define a concave recess and the brace surface 904 may comprise a concave surface bounding the concave recess. The surface 906 may comprise a convex surface disposed in the concave recess, at least in part.

Unless stated otherwise, the term “concave” means curving inward in one or more directions and/or extending inward in one or more directions. In the illustrated embodiment, the surface 904 extends inward (e.g., relative to one or more portions of the display 112) in one or more directions. A concave surface may or may not have a surface with a radius. Thus, in some embodiments, a convex surface may include one or more linear and/or planar segments. One or more of the one or more linear and/or planar segments may be similar, to one or more lines segments of a concave polygon.

Unless stated otherwise, the term “convex” means curving outward in one or more directions and/or extending outward in one or more directions. In the illustrated embodiment, the surface 906 extends outward (e.g., relative to one or more portions of the housing 102) in one or more directions. A convex surface may or may not have a surface with a radius. Thus, in some embodiments, a convex surface may include one or more linear and/or planar segments. One or more of the one or more linear and/or planar segments may be similar, to one or more lines segments of a convex polygon.

In at least some other embodiments, the brace surface 904 may comprise a convex surface and the surface 906 may comprise a concave surface.

In at least some embodiments, the interface between the brace surface 904 and the surface 906 may comprise a press fit, which may increase the friction force that resists rotation of the housing 102 relative to the display 112. In at least some of such embodiments, a convex surface may be press fit into a concave recess and/or a surface defining a concave recess may be press fit over a convex surface.

In at least some embodiments, including but not limited to the illustrated embodiment, the brace may be disposed between the display 112 and the housing 102, at least in part.

Unless stated otherwise, the term “between” means “between at least in part.” Thus, the term “between” includes “partially between” and “fully between.” Also, unless stated otherwise, the term “between” does not require adjacency and thus does not preclude the presence of additional elements and/or space.

In at least some embodiments, including but not limited to the illustrated embodiment, the brace 902 may be integral with, disposed on (directly and/or indirectly) or otherwise attached (directly and/or indirectly) to the display 112 (a housing thereof or otherwise). In at least some other embodiments, the brace may be integral with, disposed on (directly and/or indirectly) or otherwise attached (directly and/or indirectly) to the housing 102.

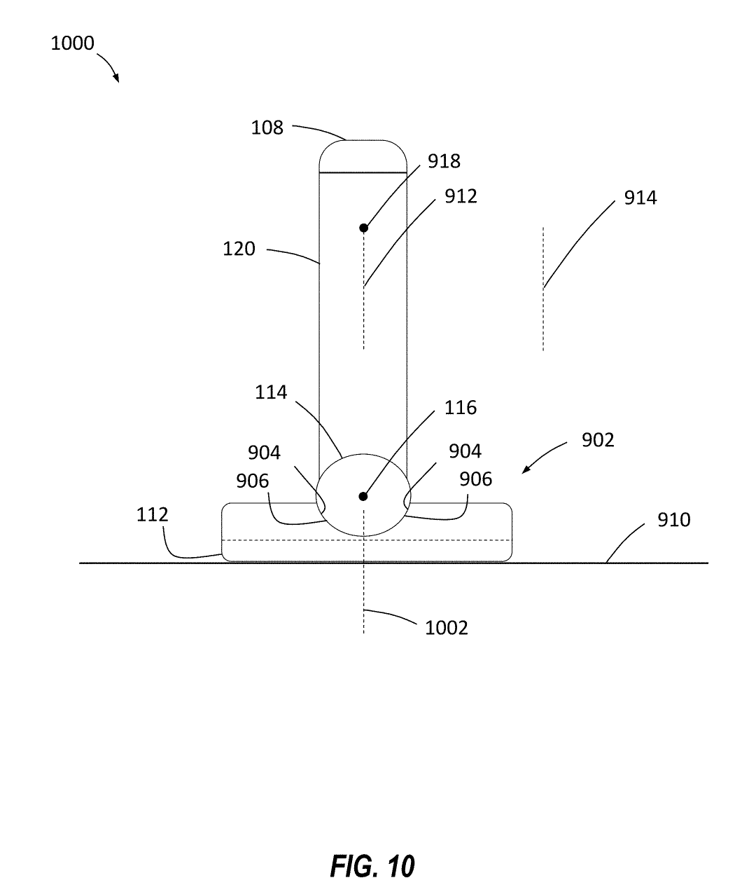

FIG. 10 shows a schematic illustration of a handheld ultrasound-imaging device 1000 that may be the same as and/or similar to the handheld ultrasound-imaging device 900 disclosed above, except that the pivot 114 and the axis 116 are disposed along (or at least nearer) a midline 1002 of the display 112, in accordance with at least some embodiments.

In at least some embodiments, the pivot 114 and/or the axis 116 may be disposed substantially along the midline 1002.

As used herein, the term substantially along a midline 1002 means within 10% of a midline 1002 of the display 112.

In at least some other embodiments, the pivot 114 and/or the axis 116 may be located anywhere between their locations shown in FIGS. 9A-9D and their locations shown in FIG. 10. In some other embodiments, the pivot 114 and/or the axis 116 may be disposed along an end of the display 112.

The brace 902 is not limited to a brace that results in frictional force.

In at least some embodiments, the brace 902 may comprise a catch.

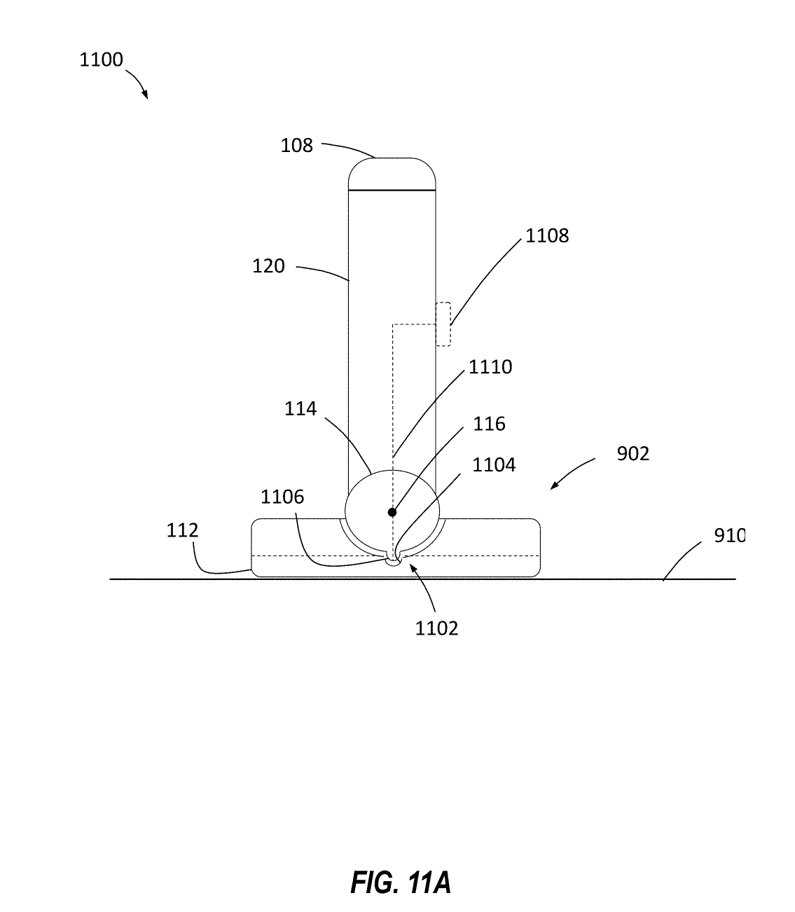

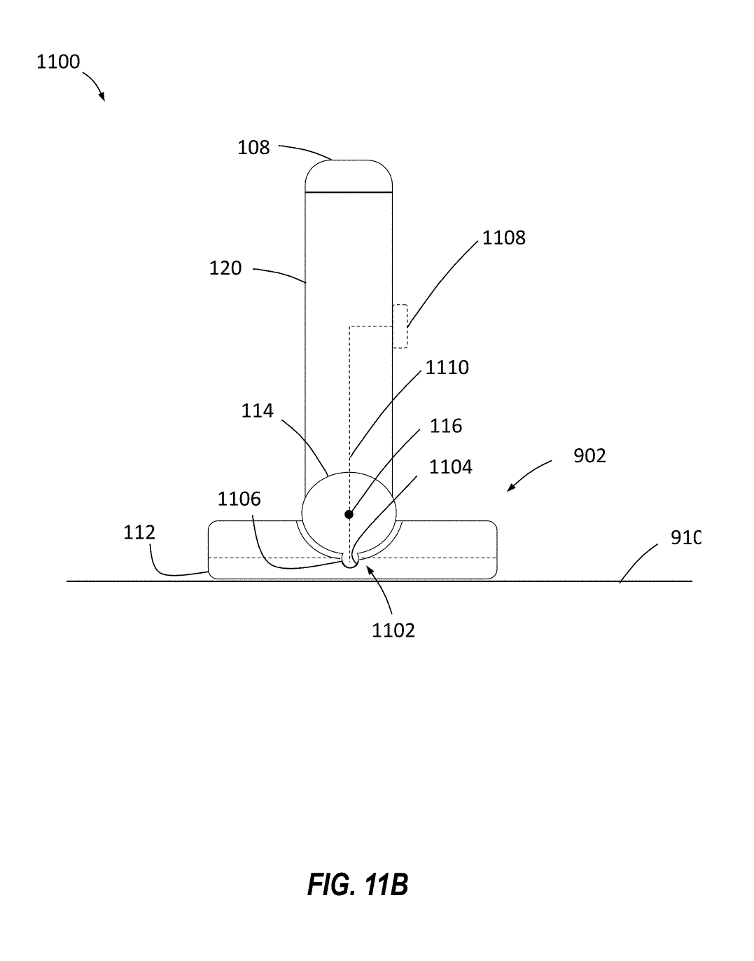

FIG. 11A shows a schematic illustration of a handheld ultrasound-imaging device 1100 that may be the same as and/or similar to the handheld ultrasound-imaging device 900 and/or the handheld ultrasound-imaging device 1000 disclosed above, except that the handheld ultrasound-imaging device 1100 includes a brace 902 that includes a catch 1102 that is releasably engageable with the housing 102 in an at least partly upright position and resists (to at least some degree) movement of the housing out of the upright position, in accordance with at least some embodiments.

Referring to FIG. 11A, in accordance with at least some embodiments, the catch 1102 may include a recess (or other concave portion) or other first feature 1104 and a projection (or other convex portion) or other second feature 1106 (complementary or otherwise to the first feature 1104) that moves relative to the first feature 1104 with relative movement between the housing 102 and the display 112, and may releasably engage the first feature 1104 with the housing in an upright position relative to the display to thereby create an interference to restrict (at least in part) relative movement between the housing 102 and the display 112.

In at least some embodiments, including but not limited to the illustrated embodiment, there is a clearance between the first feature 1104 and the second feature 1106 when the catch 1102 is engaged so as to facilitate easy operation of the catch 1102. The presence of clearance may allow some relative movement between the housing 102 and the display 112. Thus, in at least some embodiments, the catch 1102 may restrict relative movement between the housing 102 and the display 112 to a reduced range (compared to when the catch 1102 is not engaged), but need not entirely prevent relative movement between the housing 102 and the display 112.

In at least some embodiments, the catch 1102 restricts the housing to at least partly upright positions relative to the display, which in at least some embodiments, may be a sufficient amount of restriction to allow a person to apply a sterile cover. In at least some embodiments, the catch 1102 restricts relative rotation between the housing and the display to +/−45 degrees, which in at least some embodiments, makes it easier for a person to apply a sterile cover. In at least some embodiments, the catch 1102 restricts the relative rotation to +/−30 degrees, which in at least some embodiments, makes it still easier for a person to apply a sterile cover. In at least some embodiments, the catch 1102 restricts relative rotation to +/−10 degrees, which in at least some embodiments, makes it still easier for a person to apply a sterile cover. In at least some embodiments, the catch 1102 restricts relative rotation to +/−5 degrees, which in at least some embodiments, makes it still easier for a person to apply a sterile cover. In at least some embodiments, the catch 1102 restricts relative rotation to +/−1 degree, which in at least some embodiments, makes it still easier for a person to apply a sterile cover.

In at least some embodiments, there is no clearance between the first feature 1104 and the second feature 1106 so as to result in an interference that entirely prevents relative rotation between the housing 102 and the display 112.

FIG. 11B shows a schematic illustration of one such embodiment of the handheld ultrasound-imaging device 1100 in which there is no clearance between the first feature 1104 and the second feature 1106 so as to result in an interference that entirely prevents relative rotation between the housing 102 and the display 112.

In at least some embodiments, including but not limited to the illustrated embodiment, the first feature 1104 and second feature 1106 may define a detent, which may be releasable by a user or otherwise merely by application of a sufficient magnitude force to the housing 102 and/or the display 112.

In at least some embodiments, including but not limited to the illustrated embodiment, the handheld ultrasound-imaging device 1100 may optionally include a button or other manual control 1108 (shown in dashed line) mechanically coupled 1110 to the catch and activatable by a user or otherwise to release the releasably engageable catch 1102.

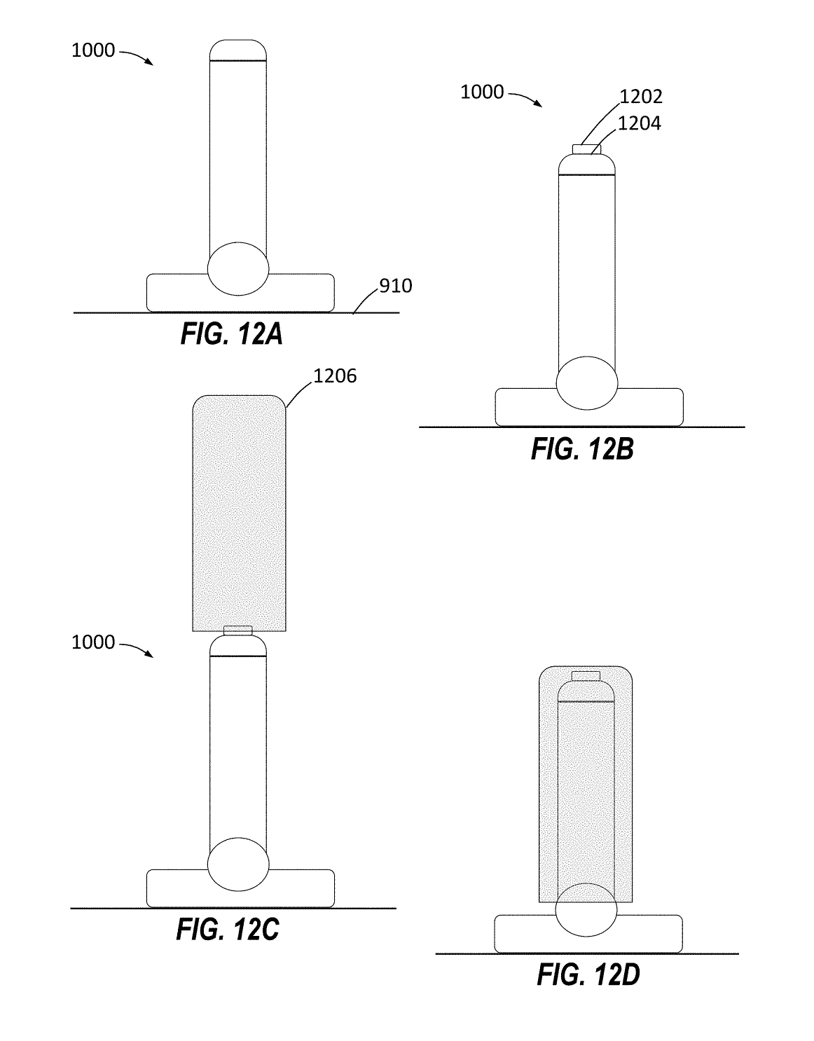

FIGS. 12A-12D illustrate schematically a method 1200 that may be used to apply a sterile cover to the handheld ultrasound-imaging device 1000 or other handheld ultrasound-imaging device, in accordance with at least some embodiments.

Referring to FIG. 12A, the method may include positioning a handheld ultrasound imaging device having a housing and a display on a surface with a screen of the display facing toward the surface, and providing relative movement between the housing and display to position an ultrasonic transducer of the handheld ultrasound imaging device in an (at least partly) upright position.

Referring to FIG. 12B, the method may further include applying an ultrasonic gel 1202 and/or other transmission medium on a membrane 1204 (or other surface) of the ultrasonic transducer.

Referring to FIGS. 12C-12D, the method may further include applying a sterile cover 1206 on at least a portion of the ultrasonic transducer. Because the brace restricts (at least in part) relative movement between the housing and the display, in at least some embodiments, 1206 may be performed without touching the handheld ultrasound imaging device.

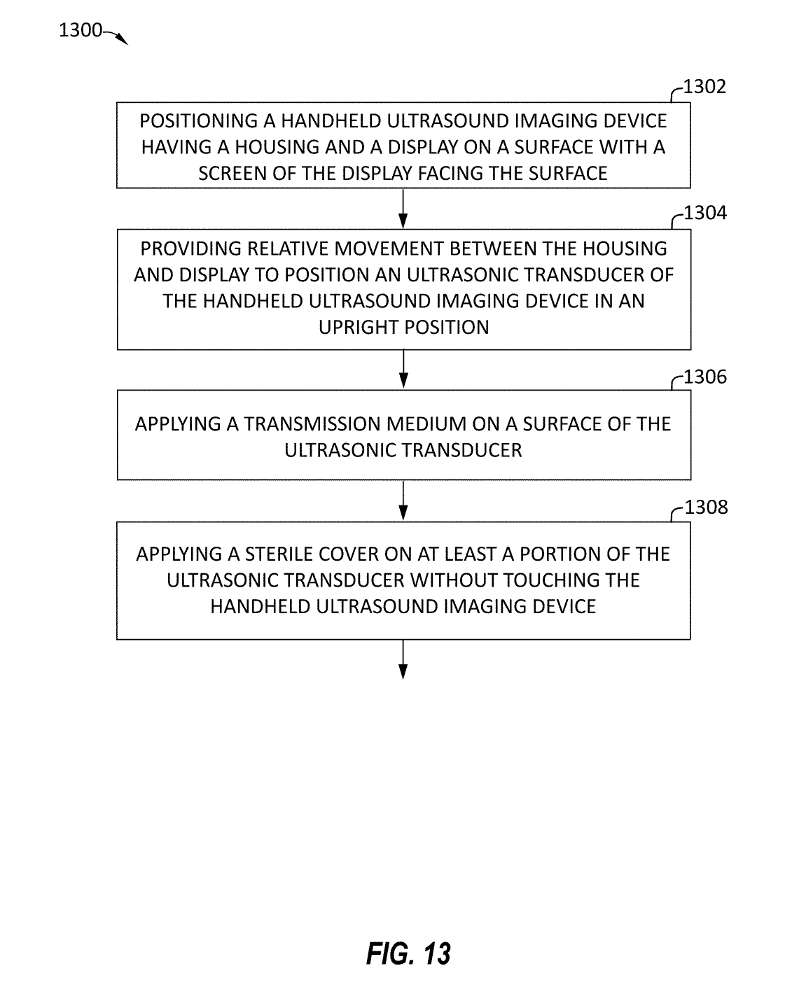

FIG. 13 is a flowchart of a method 1300, in accordance with at least some embodiments.

In at least some embodiments, the method 1300 may be used in association with one or more of the handheld ultrasound-imaging devices 900, 1000 and 1100. However, the method 1300 is not limited to such.

The method is not limited to the order shown, but rather may be performed in any practicable order. For that matter, any method disclosed herein is not limited to any particular order but rather may be performed in any practicable order.

One or more portions of the method may be used without one or more other portions of the method. For that matter, one or more portions of any method (or system) disclosed herein may be used without one or more other portions of such method (or system).

One or more portions of the method may be performed using one or more portions of one or more other methods disclosed herein.

One or more portions of the method may be performed in performance of one or more portions of one or more other methods disclosed herein.

Referring to FIG. 13, at 1302, the method may include positioning a handheld ultrasound imaging device having a housing and a display on a surface with a screen of the display facing toward the surface.

At 1304, the method may further include providing relative movement between the housing and display to position an ultrasonic transducer of the handheld ultrasound imaging device in an (at least partially) upright position.

At 1306, the method may further include applying a transmission medium on a surface of the ultrasonic transducer.

At 1308, the method may further include applying a sterile cover on at least a portion of the ultrasonic transducer. Because the brace restricts (at least in part) relative movement between the housing and the display, in at least some embodiments, 1308 may be performed without touching the handheld ultrasound imaging device.

A sterile cover may be any type of sterile cover. In at least some embodiments, the sterile cover may comprise a sterile sleeve and/or a sterile sheath. In at least some embodiments, the sterile cover may comprise one or more embodiments of the cover body 304 and/or cover 312 (which may include marking unit 302) and/or any other cover disclosed herein.

Although various embodiments of the handheld ultrasound-imaging devices 900, 1000 and 1100 and method 1200 are disclosed in association with the handheld ultrasonic ultrasound imaging device 100, the handheld ultrasound-imaging devices 900, 1000 and 1100 and this disclosure in general are not limited to such configuration. Any practicable configuration(s) of a handheld ultrasound-imaging device may be employed.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A handheld ultrasound imaging device, comprising:
a housing;
an ultrasound imaging unit disposed at least in part within the housing, the ultrasound imaging unit including an ultrasound transducer;
a display rotatably coupled to the housing; and
a releasably engageable catch;
wherein when the catch is releasably engaged, the catch restricts movement of the housing relative to the display.

2. The handheld ultrasound imaging device of claim 1, wherein when the catch is releasably engaged, the catch restricts the housing to at least partly upright positions relative to the display.

3. The handheld ultrasound imaging device of claim 1, wherein when the catch is releasably engaged, the catch resists all movement of the housing relative to the display.

4. The handheld ultrasound imaging device of claim 1, wherein the catch comprises a detent.

5. The handheld ultrasound imaging device of claim 4, wherein the catch is release by application of catch release force to at least one of the housing and the stand.

6. The handheld ultrasound imaging device of claim 1, further comprising a button coupled to the catch and depressible to release the catch.

7. A handheld ultrasound imaging device, comprising:
a housing;
an ultrasound imaging unit disposed at least in part within the housing, the ultrasound imaging unit including an ultrasound transducer;
a display;
a pivot, the housing and display rotatably coupled through the pivot; and
a brace external to said pivot to provide a friction force that resists rotation between the housing and the display.

8. The handheld ultrasound imaging device of claim 7, wherein the brace has a brace surface external to said pivot and in contact with a surface that moves relative to the brace surface with rotation between the housing and the display to provide the friction force that resists rotation between the housing and the display.

9. The handheld ultrasound imaging device of claim 7, wherein the brace surface and the surface that moves relative to the brace surface define an interface having a press fit.

10. The handheld ultrasound imaging device of claim 7, wherein the brace surface comprises a concave surface and wherein the surface that moves relative to the brace surface comprises a convex surface.

11. A method comprising:
positioning a handheld ultrasound imaging device having a housing and a display on a surface with a screen of the display facing toward the surface;
providing relative movement between the housing and display to position an ultrasonic transducer of the handheld ultrasound imaging device in an at least partially upright position; and
applying a sterile cover on at least a portion of the ultrasonic transducer without touching the handheld ultrasound imaging device.

12. The method of claim 11, further comprising applying a transmission medium on a surface of the ultrasonic transducer.
